(19) United States
(12) Patent Application Publication
(10) Pub. No.: US 2011/0052581 A1
(43) Pub. Date: Mar. 3, 2011

(54) USE OF PICOPLATIN AND CETUXIMAB TO TREAT COLORECTAL CANCER

(75) Inventors: David A. Karlin, Los ALtos, CA (US); Ronald A. Martell, San Francisco, CA (US)

(73) Assignee: Poniard Pharmaceuticals Inc., Seattle, WA (US)

(21) Appl. No.: 12/866,706

(22) PCT Filed: Feb. 6, 2009
(86) PCT No.: PCT/US09/00773

§ 371 Date: Nov. 15, 2010

Related U.S. Application Data
(60) Provisional application No. 61/027,360, filed on Feb. 8, 2008.
(60) Provisional application No. 61/027,382, filed on Feb. 8, 2008.
(60) Provisional application No. 61/027,387, filed on Feb. 8, 2008.

(51) Int. Cl. A61K 39/395; C12Q 1/02; A61P 35/00

(52) U.S. Cl. 424/133.1; 435/29

(57) ABSTRACT

The invention provides a method of treatment of metastatic colorectal cancer by administration of the anti-cancer platinum drug picoplatin in conjunction with cetuximab, 5-FU, and leucovorin in a variety of treatment regimens. The invention also provides a use of picoplatin in conjunction with cetuximab, 5-FU, and leucovorin for treatment of metastatic colorectal cancer. The invention further provides kits adapted for administration of picoplatin in conjunction with cetuximab. Also, methods for determining dosage regimens for patients afflicted with a cancer comprising EGFR are provided.

### CROSS REFERENCE TO RELATED APPLICATIONS

This application claims the priority of U.S. Ser. No. 61/027,387, filed Feb. 8, 2008, U.S. Ser. No. 61/027,382, filed Feb. 8, 2008, and U.S. Ser. No. 61/027,360, filed Feb. 8, 2008, the disclosures of which are incorporated herein by reference in their entireties. This application also claims the priority of U.S. Ser. No. 60/857,066 (filed Nov. 6, 2006), 60/857,725 (filed Nov. 8, 2006), 60/877,495 (filed Dec. 28, 2006), 60/889,191 (filed Feb. 9, 2007), 60/931,589 (filed May 24, 2007), and 60/983,852 (filed Oct. 30, 2007), and of U.S. Ser. No. 11/982,841, filed Nov. 5, 2007, the disclosures of which are incorporated by reference herein in their entireties.

### BACKGROUND OF THE INVENTION

Colorectal cancer remains the second most common cause of cancer-related death in the United States and a significant cause of cancer-related death in other countries as well.¹ For decades, the only approved chemotherapeutic drug for treatment of colorectal cancer was 5-fluorouracil (5-FU), and it continues to be the backbone of most first-line chemotherapeutic regimens for patients with advanced disease. However, there has been much progress made in treatment of metastatic colorectal cancer (mCRC) in the past decade, with the approval of several new therapeutic agents including irinotecan, oxaliplatin, capecitabine, and most recently, cetuximab and bevacizumab.^{2,3} Importantly, a variety of new chemotherapeutic regimens utilizing these agents have been devised, which have led to increased response rates and incremental increases in the time to progression and median survival for patients with advanced disease.^{2,3} Response rates for 5-FU/leucovorin, irinotecan, and oxaliplatin as single agent therapy have been low (23%, 18%, and 12%, respectively), progression-free survival has been short (median 4.0, 4.3, and 4.0 months, respectively), and median survival has also been short, approximately (12, 12, and 14.5 months, respectively).⁴ With the introduction of 5-FU-based combination chemotherapeutic regimens using irinotecan and oxaliplatin, “FOLFOX regimens,” the response rate has increased substantially, with response rates reported as high as 64% (FOLFOX7), time to progression ranging from 8.9-12.3 months, and median survival now approaching approximately 20 months in some reports.²⁻⁴

Unfortunately, however, these newer combination chemotherapy regimens do have increased toxicity. Regimens containing irinotecan are associated with significant diarrhea and other gastrointestinal toxicity, while those containing oxaliplatin are associated with neurotoxicity.²⁻¹⁰ The neurotoxicity observed is of two types: first, a cumulative and often dose limiting sensory loss with paresthesias that can interfere with function and second, a disturbing cold sensitivity that limits patient acceptance of the FOLFOX regimen.⁷⁻¹⁰

Picoplatin is a platinum analogue that has demonstrated synergy with 5-FU in vitro in pre-clinical studies and has undergone extensive Phase 1 and 2 testing in a variety of cancers.¹¹⁻²² Like other platinum analogues, picoplatin causes cell death by the formation of covalent cross-links in DNA that interfere with DNA replication and transcription, leading to cell death. Cisplatin, the first platinum analogue, was introduced approximately 20 years ago and is still widely used. The approval of cisplatin was followed by approval of carboplatin, and most recently by that of oxaliplatin.

Treatment with platinum analogues is limited by their toxicity. While neurotoxicity and nephrotoxicity are the main dose-limiting toxicities (DLT) observed following cisplatin treatment, myelosuppression is most significant following carboplatin treatment. Carboplatin is known to cause cumulative dose-related toxicity that results in slow bone marrow recovery. Peripheral neurotoxicity is well documented in patients treated with oxaliplatin. The unacceptable nephrotoxicity, oto-, and neurotoxicity associated with earlier platinum analogues has not been reported with picoplatin either in animal studies or in clinical trials.^{11, 19-22}

The efficacy of platinum analogues is also limited by several (intrinsic or acquired) mechanisms of resistance, including impaired cellular uptake, intracellular inactivation by thiols [e.g., reduced glutathione], and enhanced DNA repair and/or increased tolerance to platinum-DNA adducts.²³ Pre-clinical studies indicate that picoplatin can overcome these three mechanisms of resistance. This has been demonstrated in vitro and by using human ovarian xenograft tumor models that exhibit resistance to cisplatin.¹³⁻¹⁷ Several human ovarian and colon cell lines with induced resistance to oxaliplatin retain sensitivity to picoplatin.¹⁶⁻¹⁸

In Phase 1 studies, tolerable side-effects and indications of activity were seen in subjects with ovarian cancer, non-small cell lung cancer (NSCLC), small cell lung cancer (SCLC), colorectal cancer, head and neck cancer, renal cell cancer, thymic cancer, pancreatic cancer, stomach cancer, leiomyosarcoma, liver cancer, mesothelioma, and prostate cancers.^{24,25} In Phase 2 studies, indications of efficacy were seen in subjects with ovarian, NSCLC, SCLC, mesothelioma, prostate cancer, and breast cancer.

Cetuximab is a recombinant human/mouse chimeric epidermal growth factor receptor (EGFR) monoclonal antibody. It was approved by the U.S. Food and Drug Administration in February 2004 to be used in combination with irinotecan for the treatment of EGFR-expressing, metastatic colorectal cancer in patients who had failed to improve with irinotecan-based or oxaliplatin-based chemotherapy. Cetuximab was also approved for administration as a single agent in the treatment of patients with EGFR-expressing, metastatic colorectal cancer who are intolerant to irinotecan-based chemotherapy. It is marketed by Bristol-Myers Squibb under the brand name of Erbitux®.

EGFR is well known to be expressed in a wide variety of cancer lines, and it has been reported that “[e]levated levels of the epidermal growth factor receptor (EGFR), a growth-factor-receptor tyrosine kinase, and/or its cognate ligands have been identified as a common component of multiple cancer types and appear to promote solid tumour growth.” For example, see Nicholson R I, Gee J M, Harper M E, “EGFR and Cancer Prognosis,” Eur. J. Cancer, (September 2001), 37 Suppl. 4, S9-15.

Cetuximab has been approved as a first-line therapy used in combination with oxaliplatin and irinotecan-based regimens and as second-line therapies in combination with other drugs or as monotherapies for the treatment metastatic colorectal cancer (mCRC), for example see J. J. Lee et al., Clin Colorectal Cancer. 2007; 6 Suppl 2:S42-6; and W. Zhang et al., Ann Med. 2006; 38:545-51.

About 40% of patients with mCRC have K-ras mutations and their mCRC does not respond to epidermal growth factor receptor (EGFR) inhibitors such as cetuximab and panitumumab. Many cetuximab-treatment studies in mCRC demonstrated very low or even zero response rates, short progression-free survival, and short overall survival in K-ras mutation positive mCRC. Because K-ras wild type CRC patients treated with EGFR inhibitors have significantly higher objective response rates, increased progression-free survival, and increased overall survival, K-ras testing is now used in routine clinical practice to select the subset of mCRC patients most likely to benefit from treatment with an EGFR inhibitor. Subset selection spares patients who are unlikely to respond to EGFR inhibitors for side effects and the cost of an ineffective drug. Examples of companies that offer K-ras testing to medical oncologists include:

For example, see: M. Brink et al., Carcinogenesis. 2003; 24:703-10; A. Lièvre et al., J Clin Oncol. 2008; 26:374-9; W. De Roock et al., Ann Oncol. 2007, Nov. 12; F. Di Fiore et al., Br J Cancer. 2007; 96:1166-9; A. Lièvre et al., Cancer Res. 2006; 66:3992-5; C. S. Karapetis et al., NEJM. 2008; 359 (N 17):1757-1765; Amado et al., 2008 American Society of Clinical Oncology Gastrointestinal Cancers Symposium, Abstract 278.

### SUMMARY OF THE INVENTION

The present invention is directed to methods of treatment of metastatic colorectal cancer with picoplatin, cetuximab (Erbitux®) and optionally with 5-fluorouracil (5-FU) and leucovorin; to the use of picoplatin in conjunction with cetuximab, and optionally 5-FU and leucovorin in the treatment of colorectal cancer; and to kits adapted for administration of picoplatin in conjunction with cetuximab, 5-FU and leucovorin.

In various embodiments, the invention provides a method of treatment of colorectal cancer, comprising administering to a patient afflicted with colorectal cancer picoplatin, cetuximab, 5-fluorouracil (5-FU) and leucovorin, wherein 5-FU and leucovorin are administered intravenously at least twice at intervals of about 2-6 weeks, the picoplatin is administered with the leucovorin and 5-FU every other time that the fluorouracil and leucovorin are administered, and the cetuximab is administered at least twice at one-week intervals. For example, the picoplatin can be administered at a dose of about 60-180 mg/m², preferably at a dose of about 150 mg/m². For example, the interval of administration of the 5-FU and the leucovorin can be about two weeks and the interval of administration of the picoplatin can be about four weeks.

In various embodiments, the invention provides a method of treatment of colorectal cancer, comprising administering to a patient afflicted with colorectal cancer effective amounts of a combination of picoplatin, cetuximab, 5-FU and leucovorin, wherein the picoplatin, and the 5-FU and the leucovorin are administered intravenously at least twice at intervals of about 2-6 weeks, and the cetuximab is administered at least twice at one-week intervals, wherein an amount of picoplatin administered is less than the maximum tolerated dose of picoplatin. For example, the picoplatin can be administered at a dose of about 45-150 mg/m², preferably at a dose of about 135-150 mg/m². For example, the interval of administration of the picoplatin, 5-FU and the leucovorin can be about two weeks.

In various embodiments, the invention provides a method for selecting a treatment regimen for metastatic colorectal cancer (mCRC) comprising (a) providing a patient afflicted with mCRC; (b) determining if the patient is a K-ras wild type mCRC patient; and (c) if the patient comprises K-ras wild type mCRC, selecting for said patient a regimen comprising a EGFR inhibitor and picoplatin.

In various embodiments, the invention provides a method of treatment of colorectal cancer comprising (a) identifying a patient afflicted with colorectal cancer who has failed FOLFOX-4 and/or FOLPI regimens; and (b) administering about 5-150 mg/m² picoplatin to the patient every 21 days in combination with a first dose of 400 mg/m² cetuximab followed by a 250 mg/m² dose of cetuximab administered every week.

In various embodiments, the invention provides a method of treatment of colorectal cancer comprising (a) identifying a patient afflicted with colorectal cancer who has received irinotecan, FOLFOX, or FOLPI regimens, with or without bevacizumab or cetuximab, wherein the cancer is in remission, and (b) administering about 5-150 mg/m² picoplatin to the patient every 21 days in combination with a first dose of 400 mg/m² cetuximab followed by a 250 mg/m² dose of cetuximab administered every week as an adjuvant therapy to prevent recurrence.

In various embodiments, the invention provides a method for selecting a regimen of treatment for a patient afflicted with a metastatic cancer that comprises EGFR, comprising (a) identifying a patient afflicted with a metastatic cancer, (b) determining if the cancer comprises a wild-type K-ras gene or a mutation-positive K-ras gene and (c) selecting a treatment regimen comprising picoplatin and an EGFR inhibitor if the wild type K-ras gene is present, or selecting a treatment regimen comprising picoplatin without an EGFR inhibitor if the mutation-positive K-ras gene is present. For example, the metastatic cancer that comprises EGFR can comprise SCLC, NSCLC, a pancreatic cancer, a colorectal cancer, an epithelial cancer, or a head and neck, ovarian, cervical, bladder, esophageal, gastric, breast, or endometrial cancer.

In various embodiments, the invention provides a method for selecting a regimen of treatment for a patient afflicted with mCRC comprising: (a) identifying a patient afflicted with mCRC, (b) determining if the mCRC comprises a wild type K-ras gene or a mutated K-ras gene and (c) if the m-CRC comprises a K-ras wild type genotype, then administering to the patient an EGFR inhibitors such as cetuximab, erlotinib or panitumumab, in combination with picoplatin and, optionally, 5-FU and leucovorin, or (d) if the mCRC comprises a K-ras mutation positive genotype, then administering to the patient picoplatin and, optionally, 5-FU and leucovorin. For example, the EGFR inhibitor can comprise cetuximab.

In various embodiments, the invention provides a use of picoplatin in conjunction with cetuximab, 5-fluorouracil (5-FU), and leucovorin to treat colorectal cancer, wherein the 5-FU and leucovorin are administered intravenously at least twice at intervals of about 2-6 weeks, the picoplatin is administered with the leucovorin and 5-FU every other time that the fluorouracil and leucovorin are administered, and the cetuximab is administered at least twice at one-week intervals. For example, the picoplatin can be administered at a dose of about 60-180 mg/m², preferably at a dose of about 150 mg/m². For example, the interval of administration of the 5-FU and the leucovorin can be about two weeks and the interval of administration of the picoplatin can be about four weeks.

In various embodiments, the invention provides a use of picoplatin in conjunction with cetuximab, 5-FU and leucovorin, wherein the picoplatin, and the 5-FU and the leucovorin are administered intravenously at least twice at intervals of about 2-6 weeks, and the cetuximab is administered at least twice at one-week intervals, wherein an amount of picoplatin administered is less than the maximum tolerated dose of picoplatin. For example, the picoplatin can be administered at a dose of about 45-150 mg/m², preferably at a dose of about 135-150 mg/m². For example, the interval of administration of the picoplatin, 5-FU and the leucovorin can be about two weeks.

In various embodiments of the invention, a kit is provided, the kit being adapted for the intravenous administration of a FOLPI plus cetuximab regimen to a patient; the kit comprising a first container comprising a solution of picoplatin and a second container comprising a solution of leucovorin; further comprising a coupling adapted to be independently connected to the first container, the second container, and a single intravenous tube, so that the content of the first container and the second container can be simultaneously administered to the patient; the kit further comprising a container comprising a solution of cetuximab (Erbitux®) and a container comprising a solution of 5-FU, adapted for intravenous administration to the patient; optionally further comprising instructions for use.

In an embodiment, the kit can include, in the first container, picoplatin in a dosage form comprising an isotonic solution comprising water, a tonicity adjuster comprising NaCl, and about 0.5 mg/mL dissolved picoplatin. The dosage form can also comprise an effective amount of dissolved or dispersed 5-FU and/or leucovorin in accord with the doses disclosed herein. The dosage form also does not contain a preservative or bacteriostatic agent. An appropriate volume of the dosage form can be administered to achieve a desired therapeutic dose.

### DETAILED DESCRIPTION OF THE INVENTION

In various embodiments, the invention provides a method of treatment of colorectal cancer, comprising administering to a patient afflicted with colorectal cancer picoplatin, cetuximab, 5-fluorouracil (5-FU) and leucovorin, wherein 5-FU and leucovorin are administered intravenously at least twice at intervals of about 2-6 weeks, the picoplatin is administered with the leucovorin and 5-FU every other time that the fluorouracil and leucovorin are administered, and the cetuximab is administered at least twice at one-week intervals. For example, the picoplatin can be administered at a dose of about 60-180 mg/m², preferably at a dose of about 150 mg/m². For example, the interval of administration of the 5-FU and the leucovorin can be about two weeks and the interval of administration of the picoplatin can be about four weeks.

In various embodiments, the invention provides a method of treatment of colorectal cancer, comprising administering to a patient afflicted with colorectal cancer effective amounts of a combination of picoplatin, cetuximab, 5-FU and leucovorin, wherein the picoplatin, and the 5-FU and the leucovorin are administered intravenously at least twice at intervals of about 2-6 weeks, and the cetuximab is administered at least twice at one-week intervals, wherein an amount of picoplatin administered is less than the maximum tolerated dose of picoplatin. For example, the picoplatin can be administered at a dose of about 45-150 mg/m², preferably at a dose of about 135-150 mg/m². For example, the interval of administration of the picoplatin, 5-FU and the leucovorin can be about two weeks.

For example, the leucovorin and the 5-FU can be administered about every two weeks, the picoplatin is administered with the leucovorin about every 4 weeks, and the cetuximab is administered weekly. For example, the picoplatin can be administered at least once at a dosage of about 60-75 mg/m². Alternatively the picoplatin can be administered at least once at a dose of about 150 mg/m². In some embodiments, a subsequent dose of picoplatin can be administered at about a 15-30 mg/m² lower dose than a previous dose.

In various embodiments of the inventive method, the patient has not previously been treated for metastatic disease. In other embodiments, the patient can have previously been treated with an irinotecan, FOLFOX and/or FOLPI regimen. Or, the patient can have previously been treated with a FOLFOX regimen, and subsequently with a FOLPI regimen and has relapsed within 6 months of completing the FOLPI regimen. Alternatively, the patient can have previously been treated with a first regimen comprising FOLFOX or irinotecan, and subsequently with a second regimen comprising cetuximab alone, irinotecan plus cetuximab, or FOLPI, and has relapsed within 6 months following cessation of the second regimen.

In various embodiments of the present method, the patient has not previously been treated for metastatic disease, or the patient has not previously had systemic treatment, such as chemotherapy, for localized or metastatic disease. For example, the patient may have had surgery to remove or to de-bulk the primary tumor and then be treated with one of the picoplatin, 5-FU, leucovorin regimens (e.g., FOLPI) of the invention to prevent or delay progression of the cancer, including to prevent or delay the development of metastases. The patient may have received earlier chemotherapy at the time of primary tumor treatment, at least 6 months prior to the present picoplatin treatment.

In various embodiments, the picoplatin can be administered with curative intent, rather than merely seeking to arrest the disease with no remission. The dosage of the picoplatin can be increased beyond that bringing about disease stasis in order to achieve a cure in the patient.

For example, the picoplatin can be administered substantially concurrently with the leucovorin followed by administration of the 5-FU at every treatment of the patient, and the cetuximab is administered at one week intervals. For example, the picoplatin can be administered at least once at a dosage of about 40-45 mg/m².

In various embodiments, the patient can be previously been treated with an earlier systemic regimen of chemotherapy and the cancer be in remission. For example, the patient can have been treated with an earlier FOLPI regimen, with or without bevacizumab or cetuximab.

In various embodiments of the inventive method, the picoplatin can be administered in a dosage form comprising an isotonic solution comprising water, a tonicity adjuster comprising NaCl, and about 0.5 mg/mL dissolved picoplatin, wherein the dosage form does not contain a preservative or bacteriostatic agent.

In various embodiments, the picoplatin, the cetuximab and the leucovorin can be administered substantially concurrently. For example, the picoplatin and the leucovorin can be administered simultaneously. As used herein, the term “concurrently” means that the administrations are simultaneous, overlapping or close enough in time so that the two or more agents administered are present in vivo in therapeutically effective amounts.

In various embodiments, the 5-FU can be administered following the administration of the picoplatin, leucovorin and cetuximab.

In various embodiments, the leucovorin can be administered at an initial dosage of about 200-400 mg/m². And, the 5-FU can be administered at a total dosage per dosing of about 1000-3000 mg/m².

In various embodiment, the picoplatin can be administered at a dosage of about 60-180 mg/m². More specifically, the picoplatin can administered at a dosage of about 120-150 mg/m². For example, the picoplatin can be administered at least once at a dosage of about 150 mg/m².

In various embodiments, a subsequent dose of picoplatin can be administered at about a 15-30 mg/m² lower dose than a previous dose; for example when the previous dose is about 150 mg/m², the subsequent dose can be about 120-135 mg/m².

In various embodiments of the inventive method, a cumulative dose of greater than about 900 mg/m² of picoplatin is delivered to the patient.

In various embodiments, the cetuximab can be administered intravenously at a first dose of about 400 mg/m², then once a week at a dose of about 250 mg/m².

In various embodiments, the leucovorin, at a dosage of about 400 mg/m², can be administered as a 2 hour infusion, the administration of the leucovorin being followed by a 5-FU bolus at a dosage of about 400 mg/m²; the 5-FU bolus being followed by 5-FU at a dosage of about 2,400 mg/m² administered as a 46 hour continuous infusion; wherein the leucovorin and the 5-FU are administered to the patient every 2 weeks and about 60-150 mg/m² of the picoplatin is administered to the patient with the leucovorin every 4 weeks, wherein at least the initial dose of picoplatin is about 150 mg/m², and wherein the cetuximab is administered at an initial dose of about 400 mg/m², then once a week at a dose of about 250 mg/m².

An embodiment of the inventive method provides a method of treatment of colorectal cancer comprising:

(a) identifying a patient afflicted with colorectal cancer who has failed FOLFOX-4 and/or FOLPI regimens; and

(b) administering about 5-150 mg/m² picoplatin to the patient every 21 days in combination with a first dose of 400 mg/m² cetuximab followed by a 250 mg/m² dose of cetuximab administered every week.

An embodiment of the inventive method provides a method of treatment of colorectal cancer comprising:

(a) identifying a patient afflicted with colorectal cancer who has received irinotecan, FOLFOX, or FOLPI regimens, with or without bevacizumab or cetuximab, wherein the cancer is in remission, and

(b) administering about 5-150 mg/m² picoplatin to the patient every 21 days in combination with a first dose of 400 mg/m² cetuximab followed by a 250 mg/m² dose of cetuximab administered every week as an adjuvant therapy to prevent recurrence.

In another embodiment of the invention, the picoplatin is administered substantially concurrently with the leucovorin and the picoplatin is administered at every second treatment of the patient with the 5-FU and the leucovorin, e.g., every four weeks. The cetuximab is administered in a relatively high dose concurrently with the picoplatin and then weekly thereafter. The leucovorin can be administered at a dosage of about 200-500 mg/m², preferably at about 400 mg/m². The picoplatin is administered at a dosage of about 60-180 mg/m². The cetuximab is administered by infusion at a dose of 400 mg/m² over about 20 hrs. at about 5 mL/min, followed by a weekly maintenance dose of 250 mg/m², infused i.v. over about 60 minutes. The 5-FU is administered at a total dosage of about 1000-3000 mg/m². A preferred treatment cycle for leucovorin and 5-FU is every two weeks, and picoplatin is administered every 4 weeks, e.g., at a low dose of about 60-75 mg/m², e.g., 60 mg/m², or at a high dose of about 120-180 mg/m², preferably about 120-150 mg/m², e.g. about 150 mg/m².

Therefore, in one embodiment of the invention, the leucovorin, at a dosage of 200-500 mg/m², is administered as an about 2 hour infusion concurrently with the picoplatin, when it is given, wherein the picoplatin dosage is 120-180 mg/m², e.g., about 150 mg/m²; the administration of the leucovorin and the picoplatin being followed by a 5-FU dosage of about 400 mg/m² as a bolus; the 5-FU dosage being followed by 5-FU at a dosage of 600 mg/m² or 2,400 mg/m², preferably administered as a 22 hour or as a 46 hour continuous infusion, respectively, wherein the leucovorin and 5-FU are provided to the patient at intervals of two weeks and the leucovorin, picoplatin, and 5-FU are provided to the patient at alternating intervals of four weeks. The cetuximab is administered as described above, at an initial dose of 400 mg/m² followed by weekly doses of 250 mg/m². In another embodiment, a low dose of picoplatin of about 45-75 mg/m², e.g., about 60-75 mg/m², e.g., about 60 mg/m², is administered. Such 5-FU/leucovorin/picoplatin regimens can be broadly termed FOLPI regimens which, in the present invention, are supplemented by cetuximab infusions.

In another embodiment of the invention, the leucovorin, at a dosage of 400 mg/m², is administered as a 2 hour infusion; the administration of the leucovorin being followed by a 5-FU bolus at a dosage of 400 mg/m²; the 5-FU bolus dosage being followed by parenteral 5-FU at a dosage of 400 mg/m² or 2,400 mg/m², preferably administered as a 22 hour or as a 46 hour continuous infusion, respectively; the administration of the leucovorin and the 5-FU taking place every two weeks; wherein every two weeks picoplatin, at a dosage of up to about 50 mg/m², e.g., at about 40-50 m g/m², e.g., about 45 mg/m², is administered concurrently with the leucovorin, preferably simultaneously. Picoplatin dosages of about 45-105 mg/m² can also be administered. Cetuximab is given weekly as described hereinabove.

It has unexpectedly been found that, in some cases, the combination of low doses of picoplatin administered with leucovorin and 5-FU at every treatment cycle, are as effective as, or more effective than, higher doses, e.g., the MTD, given at the same intervals, in producing a response. The MTD for the 2 week and 4 week picoplatin administration schedules (see Table 1) are discussed below. Preferably, such doses in the initial treatment are lower or substantially lower than the MTD. Such doses can range from about 40-60 mg/m² of picoplatin every two weeks, given with leucovorin and cetuximab and followed by 5-FU, as discussed below.

It has surprisingly been found that a total cumulative picoplatin dose in excess of about 900 mg/m² can be tolerated by patients without neuropathy of Grade 2 or higher being observed.

In one embodiment of the present method, the patient preferably has not previously had systemic treatment, such as chemotherapy, for metastatic disease. The patient may have, however, received earlier adjuvant therapy at the time of primary tumor treatment, at least 6 months prior to the present picoplatin-cetuximab treatment.

In another embodiment of the invention, the patient has been treated with an earlier systemic regimen of chemotherapy, such as a FOLFOX regimen and is in remission. In such cases the present regimen, broadly termed FOLPI, with or without cetuximab, can be administered to prolong the period of remission, or disease-free survival. Alternatively, the MCRC patient has been treated with an earlier FOLPI regimen, with or without bevacizumab or cetuximab, and the cancer is in remission, and a present method of FOLPI plus cetuximab, or a combination of picoplatin and cetuximab, can be used as an adjuvant therapy to prevent recurrence of the cancer.

Preferably, the patient exhibits EGFR expression in at least some of the cells of the metastatic colorectal cancer.

Picoplatin can used in combination with 5-fluorouracil (5-FU) and leucovorin in the FOLPI regimen as a first-line treatment in patients with mCRC and cetuximab can also be administered. Epidermal growth factor receptor (EDFR) inhibitors such as the monoclonal antibodies cetuximab or panitumumab are used as second- and third- line treatments in patients with mCRC. As discussed above, mCRC patients with a tumor genotype that is K-ras mutation positive are unresponsive to cetuximab and other EGFR agonists. In various embodiments of the present invention, picoplatin and cetuximab, optionally including 5-FU and leucovorin, can be used in the treatment of patients having K-ras mutation positive mCRC. For example, a patient who would otherwise receive a particular dose of cetuximab, but whose mCRC cancer cell genotype is found to be K-ras mutation positive, can be administered picoplatin in conjunction with 5-FU and leucovorin in various dosing regimens.

Thus, an embodiment of the present invention also comprises a method for selecting a regimen of treatment for a patient afflicted with mCRC comprising: (a) providing a patient afflicted with mCRC, (b) determining if the mCRC comprises a wild type K-ras gene or a mutated K-ras gene and (c) selecting a regimen for said K-ras wild type mCRC patient comprising the combination therapy of the invention described hereinabove, comprising one or more EGFR inhibitors such as cetuximab (such as Erbitux®), erlotinib (such as Tarceva®) or panitumumab (such as Vectibix®), picoplatin, 5-FU and leucovorin. If the patient is determined to comprise mCRC that is K-ras mutation positive, an EGFR inhibitor would be omitted from the picoplatin, 5-FU, leucovorin regimen. A further embodiment comprises treating said patient with the selected regimen.

The present method can generally be employed to select a regimen of treatment for a patient afflicted with a cancer, such as SCLC, NSCLC, a pancreatic cancer, a colorectal cancer, an epithelial cancer, or a head and neck, ovarian, cervical, bladder, esophageal, gastric, breast, or endometrial cancer, or the like, that comprises EGFR, by providing a patient afflicted with a tumor, (b) determining if the tumor comprises a wild-type K-ras gene or a mutated K-ras gene and (c) selecting a treatment regimen comprising picoplatin and an EGFR inhibitor if the wild type K-ras gene is present.

An embodiment of the invention also provides a method for selecting a regimen of treatment for a patient afflicted with mCRC comprising: (a) identifying a patient afflicted with mCRC, (b) determining if the mCRC comprises a wild type K-ras gene or a mutated K-ras gene and (c) if the m-CRC comprises a K-ras wild type genotype, then administering to the patient an EGFR inhibitors such as cetuximab, erlotinib or panitumumab, in combination with picoplatin and, optionally, 5-FU and leucovorin, or (d) if the mCRC comprises a K-ras mutation positive genotype, then administering to the patient picoplatin and, optionally, 5-FU and leucovorin. For example, the EGFR inhibitor can be cetuximab.

In a further embodiment of the invention, the patient afflicted with colorectal cancer has failed first line therapy (FOLFOX or irinotecan) and has failed second-line therapy as well (cetuximab alone, irinotecan plus cetuximab or FOLPI). In such cases, the present modified FOLPI plus cetuximab regimens can be employed as “third-line therapy.”

Alternatively, employing intravenous picoplatin (5-150 mg/m²) every 3 weeks in combination with the cetuximab regimen 400 mg/m² i.v. initial loading dose, then 250 mg/m² i.v. weekly maintenance doses can be employed as third-line therapy, without further administration of 5-FU and leucovorin.

As used herein, the term “concurrently” means that the administrations are simultaneous, overlapping or close enough in time so that the two or more agents administered are present in vivo in therapeutically effective amounts.

The present method also can comprise administration of an effective amount of a 5-HT₃ receptor antagonist, as an anti-emetic.

An embodiment of the invention provides a use of picoplatin in conjunction with cetuximab, 5-fluorouracil (5-FU), and leucovorin to treat metastatic colorectal cancer, wherein the 5-FU and leucovorin are administered intravenously at least twice at intervals of about 2-6 weeks, the picoplatin is administered with the leucovorin and 5-FU every other time that the fluorouracil and leucovorin are administered, and the cetuximab is administered at least twice at one-week intervals.

For example, in various embodiments the leucovorin and the 5-FU can be administered about every two weeks, the picoplatin administered with the leucovorin about every 4 weeks, and the cetuximab administered weekly. For example, the picoplatin can be administered at least once at a dosage of about 60-75 mg/m².

Another embodiment of the invention provides a use of picoplatin in conjunction with cetuximab, 5-fluorouracil (5-FU), and leucovorin to treat metastatic colorectal cancer, wherein the picoplatin, 5-FU and leucovorin are administered intravenously at least twice at intervals of about two weeks, and the cetuximab is administered at least twice at one-week intervals, wherein the amount of picoplatin is less than the maximum tolerated dose of picoplatin when administered in said combination.

Another embodiment of the invention provides a use of picoplatin in conjunction with cetuximab, 5-fluorouracil (5-FU), and leucovorin to treat metastatic colorectal cancer, wherein 5-FU and leucovorin are administered intravenously at intervals of about two weeks, and the picoplatin is administered with the leucovorin and 5-FU every time that the fluorouracil and leucovorin are administered, wherein the picoplatin is administered at a dose of about 45-120 mg/m², and wherein the cetuximab is administered intravenously at a first dose of about 250-500 mg/m², followed by doses of about 200-300 mg/m² administered at weekly intervals.

For example, the picoplatin can administered substantially concurrently with the leucovorin followed by administration of the 5-FU at every treatment of the patient, and the cetuximab administered at one week intervals. For example, the picoplatin can be administered at least once at a dosage of about 40-45 mg/m².

In various embodiments of the inventive use, the patient has not previously been treated for metastatic disease. In various other embodiments, the patient has previously been treated with a FOLFOX and/or FOLPI regimen. For example, the patient can previously have been treated with a FOLFOX regimen and subsequently with a FOLPI regimen and relapsed within 6 months of completing the FOLPI regimen. Or, the patient afflicted with colorectal cancer can have been treated with a first regimen comprising FOLFOX or irinotecan, and subsequently with a second regimen, comprising cetuximab alone, irinotecan plus cetuximab or FOLPI, and have relapsed within 6 months following cessation of the second regimen.

In other embodiments of the inventive use, the patient can have previously been treated with an earlier systemic regimen of chemotherapy and the cancer can be in remission. For example, the patient can have been treated with an earlier FOLPI regimen, with or without bevacizumab or cetuximab, and the cancer can be in remission.

In various embodiments, the picoplatin can be administered in a dosage form comprising an isotonic solution comprising water, a tonicity adjuster comprising NaCl, and about 0.5 mg/mL dissolved picoplatin, wherein the dosage form does not contain a preservative or bacteriostatic agent.

In various embodiments, the picoplatin, the cetuximab and the leucovorin can be administered substantially concurrently. As used herein, the term “concurrently” means that the administrations are simultaneous, overlapping or close enough in time so that the two or more agents administered are present in vivo in therapeutically effective amounts. In various embodiments, the picoplatin and the leucovorin can be administered simultaneously. In various embodiments, the 5-FU can be administered following the administration of the picoplatin, leucovorin and cetuximab.

In various embodiments of the inventive use, the leucovorin can be administered at an initial dosage of about 200-400 mg/m². In other embodiments, the 5-FU can be administered at a total dosage per dosing of about 1000-3000 mg/m². In various embodiments, the picoplatin can be administered at a dosage of about 60-180 mg/m². More specifically, the picoplatin can administered at a dosage of about 120-150 mg/m²; for example, the picoplatin can be administered at least once at a dosage of about 150 mg/m². In various embodiments, a subsequent dose of picoplatin can be administered at about a 15-30 mg/m² lower dose than a previous dose; for example when the previous dose is about 150 mg/m², the subsequent dose can be about 120-135 mg/m². In various embodiments, a cumulative dose of greater than about 900 mg/m² of picoplatin can be delivered to the patient.

In various embodiments, the cetuximab can be administered intravenously at a first dose of about 400 mg/m², then once a week at a dose of about 250 mg/m².

In various embodiments of the inventive use, the leucovorin, at a dosage of about 400 mg/m², can be administered as a 2 hour infusion, the administration of the leucovorin being followed by a 5-FU bolus at a dosage of about 400 mg/m²; the 5-FU bolus being followed by 5-FU at a dosage of about 2,400 mg/m² administered as a 46 hour continuous infusion; wherein the leucovorin and the 5-FU are administered to the patient every 2 weeks and about 60-150 mg/m² of the picoplatin is administered to the patient with the leucovorin every 4 weeks, wherein at least the initial dose of picoplatin is about 150 mg/m², and wherein the cetuximab is administered at an initial dose of about 400 mg/m², then once a week at a dose of about 250 mg/m².

In various embodiments, the patient can exhibit EGFR expression in at least some cells of the metastatic colorectal cancer.

In various embodiment, about 5-150 mg/m² picoplatin can be administered every 21 days in conjunction with a first dose of 400 mg/m² cetuximab followed by a 250 mg/m² dose of cetuximab administered every week in treatment of colorectal cancer in a patient afflicted with colorectal cancer who has failed FOLFOX-4 and/or FOLPI regimens.

In various embodiments, about 5-150 mg/m² picoplatin can be administered every 21 days in conjunction with a first dose of 400 mg/m² cetuximab followed by a 250 mg/m² dose of cetuximab administered every week to prevent recurrence of colorectal cancer in a patent afflicted with colorectal cancer who has received irinotecan, FOLFOX, or FOLPI regimens, with or without bevacizumab or cetuximab, wherein the cancer is in remission.

In various embodiments, the use can further comprise administration of a 5-HT₃ receptor antagonist.

In various embodiments, the invention provides a kit adapted for the intravenous administration of a FOLPI plus cetuximab regimen to a patient; the kit comprising a first container comprising a solution of picoplatin and a second container comprising a solution of leucovorin; further comprising a coupling adapted to be independently connected to the first container, the second container, and a single intravenous tube, so that the content of the first container and the second container can be simultaneously administered to the patient; the kit further comprising a container comprising a solution of cetuximab (Erbitux®) and a container comprising a solution of 5-FU, adapted for intravenous administration to the patient; optionally further comprising instructions for use. For example, the first container can comprise a dosage form of picoplatin comprising an isotonic solution comprising water, a tonicity adjuster, and about 0.5 mg/mL dissolved picoplatin, wherein the dosage form does not contain a preservative or bacteriostatic agent.

Picoplatin (SP-4-3) (cis-aminedichloro(2-methylpyridine)Pt(II)), and useful prodrugs and analogs thereof are disclosed in U.S. Pat. Nos. 5,665,771; 6,518,428; 6,413,953; U.S. patent application Ser. No. 11/982,891, filed Nov. 5, 2007; and PCT/GB/01/02060, which are incorporated herein by reference. The doses disclosed herein can be providing by oral administration of an effective amount of picoplatin in combination with a pharmaceutically acceptable vehicle, as well as by intravenous infusion.

ERBITUX® (cetuximab) is a recombinant, human/mouse chimeric monoclonal antibody that binds specifically to the extracellular domain of the human epidermal growth factor receptor (EGFR). ERBITUX® is composed of the Fv regions of a murine anti-EGFR antibody with human IgG1 heavy and kappa light chain constant regions and has an approximate weight of 152 kDa. ERBITUX® is produced in mammalian (murine myeloma) cell culture. See, Goldstein et al. (U.S. Pat. No. 7,060,808).

ERBITUX® is provided as a sterile, clear, colorless liquid of pH 7.0 to 7.4, which may contain a small amount of easily visible, white, amorphous, cetuximab particulates. Each single-use, 50-mL vial contains 100 mg of cetuximab at a concentration of 2 mg/mL and is formulated in a preservative-free solution containing 8.48 mg/mL sodium chloride, 1.88 mg/mL sodium phosphate dibasic heptahydrate, 0.42 mg/mL sodium phosphate monobasic monohydrate, and Water for Injection, USP.

ERBITUX® administered in combination with concomitant chemotherapy or radiation therapy exhibits nonlinear pharmacokinetics. The area under the concentration time curve (AUC) increased in greater than dose proportional manner while clearance of cetuximab decreased from 0.08 to 0.02 L/h/m² as the dose increased from 20 to 200 mg/m², and at doses >200 mg/m², it appeared to plateau. The volume of the distribution for cetuximab appeared to be independent of dose and approximated the vascular space of 2-3 L/m².

The recommended dose regimen is 400 mg/m² initial dose as a 120 min. intravenous infusion followed by 250 mg/m² weekly dose infused i.v. over 60 min., continued until disease progression or unacceptable toxicity. The in vitro concentrations of cetuximab reached steady-state levels by the third weekly infusion with mean peak and trough concentrations across studies ranging from 168 to 235 and 41 to 85 μg/mL, respectively. The mean half-life of cetuximab was approximately 112 hours (range 63-230 hours). The pharmacokinetics of cetuximab were similar in patients with SCCHN and those with colorectal cancer. Cetuximab has been evaluated in combination with FOLFOX 4 regimen without undue side effects.²⁷

The use of picoplatin to treat metastatic colorectal cancer will be conducted in three parts. Phase 1 is a dose escalation study to identify the maximum tolerated dose (MTD) of picoplatin that can be administered either every two weeks or every four weeks, with 5-FU and leucovorin (LV) administered every two weeks, as initial therapy for subjects with metastatic colorectal cancer who have not been previously treated for metastatic disease. Phase 2 is a randomized study. In one arm of the study, picoplatin is administered at 150 mg/m² every four weeks, combined with 5-FU and leucovorin that are administered every two weeks. In the other arm, a modified FOLFOX 6 regimen is employed wherein the 100 mg/m² oxaliplatin dose in FOLFOX 6 has been reduced to 85 mg/m², and is administered every 2 weeks, so that the two agents can be compared in the context of a widely used regimen. It is believed that cancer patients can be more effectively treated with the regimens of the present invention, which employ picoplatin instead of cisplatin, carboplatin or oxaliplatin, because they will experience fewer side effects, such as neuropathy, while preferably receiving higher doses of the platinum (Pt) drug. Phase 3 will be a study comparing the FOLPI regimen with and without weekly Erbitux® infusions.

Subjects eligible for the Phase 1 study will have Stage IV colorectal cancer and will have received no systemic therapy for metastatic cancer. Prior adjuvant chemotherapy with a 5-FU-based treatment regimen not containing oxaliplatin or irinotecan is acceptable if there has been a treatment-free interval of at least 6 months.

### Phase 1

Subjects are assigned centrally to treatment with picoplatin administered either every two or every four weeks and are assigned a dose of picoplatin to be given dependent on the study results to date. Each patient also receives 5-FU and leucovorin therapy every two weeks. Cohorts of 3 subjects receive their assigned dose of picoplatin and leucovorin and 5-FU according to the following schedule:

Day 1: Picoplatin, assigned dosage, as a 2-hour infusion, given either every cycle of 5-FU and leucovorin (q 2 weeks, Schedule A) or with every other cycle of 5-FU and leucovorin (q 4 weeks, Schedule B). Leucovorin, 400 mg/m² in D5W (water-5% dextrose), will be administered as a 2 hour infusion, either alone or, if the patient is to receive picoplatin, at the same time as picoplatin in separate bags using a Y-line. The leucovorin (±picoplatin) will be followed by a 5-FU bolus=400 mg/m² and then by 5-FU, 2,400 mg/m² in D5W administered as a 46 hour continuous infusion.

Subjects in Phase 1 are centrally assigned to one of two schedules of picoplatin. The first cohort of q 2 week (Schedule A) subjects are treated with picoplatin at a dosage of 45 mg/m², every cycle, q 2 weeks. Subsequent sequential cohorts of subjects assigned to this schedule receive picoplatin at dose levels increasing by 15 mg/m² if treatment is well tolerated and until unacceptable dose-limiting toxicity (DLT) establishes the MTD.

The MTD is defined as the dose of picoplatin below the dose at which at least one third of at least 6 subjects experience a DLT. Tolerance data from only the first 4 weeks of treatment is used to determine the MTD. Thus, data following the first two doses of picoplatin in the q 2 week (Schedule A) subjects and following only the first dose of picoplatin in the q 4 week (Schedule B) subjects are considered. The first cohort of q 4 week (Schedule B) subjects will be treated with picoplatin at a dosage of 60 mg/m², every other cycle, q 4 weeks. Subsequent sequential cohorts of subjects assigned to this schedule will receive picoplatin at dose levels increasing by 30 mg/m² if treatment is well tolerated and until unacceptable dose-limiting toxicity (DLT) establishes the MTD. Depending on the pattern and severity of toxicity observed, additional intermediate dose levels of either schedule of picoplatin administration may be studied.

Within each schedule, the cohort size is 3 subjects, and is expanded to 6 subjects if a DLT is observed. Within each cohort of each schedule, one patient is treated initially; if no DLT is observed within the following 4 weeks (2 drug cycles), the remaining two subjects may be treated. If a DLT is observed in the first patient within a cohort, whether or not to proceed with enrollment of additional subjects in the cohort will be determined on a case-by-case basis. All subjects within a q 2 week (Schedule A) cohort will have completed 2 cycles (a cycle=the 2-day treatment regimen and an additional 12-day follow-up period) prior to escalating the dose in the next cohort of subjects. All subjects within a q 4 week (Schedule B) cohort will have completed 1 cycle of the 2-day treatment regimen (which should include 5FU/leucovorin) and an additional 26-day follow-up period prior to escalating the dose in the next cohort of Schedule B subjects.

If no DLT is observed among the 3 subjects within a cohort, picoplatin dose escalation may proceed in the next cohort of that schedule of picoplatin. If one DLT is observed, the cohort size at the specified dose and schedule of picoplatin is expanded to 6 subjects. Additional subjects may be entered at any dosage level and schedule below the dose at which 2 of 6 have DLT to obtain additional safety or efficacy data.

### Phase 2

The dose of the Phase 2 component of this study is selected based on the dose intensity of picoplatin achieved on each dose and schedule, the number of cycles tolerated and a subjective assessment of the tolerability and safety profile of each dose and schedule and a preliminary assessment of response rate in accord with Phase 1. The schedule for Phase 2 is selected as Schedule B, the q 4 week schedule. The subjects (approximately 100 with metastatic CRC, at about 25 clinical sites) are randomized to the modified FOLFOX 6⁶ or to FOLPI-150.

The FOLPI regimen is as follows:

Picoplatin 150 mg/m², is administered with every alternate cycle of 5-FU and leucovorin (q 4 weeks, Schedule B) as a 2 hour infusion. Leucovorin (400 mg/m² in D5W) is administered every 2 weeks as a 2-hour infusion, either alone, or given at the same time as the picoplatin in a separate bag using a Y-line. The administration of leucovorin ±picoplatin is followed by a 5-FU bolus of 400 mg/m² and then by 5-FU, 2400 mg/m² in D5W administered as a 46 hour continuous infusion.

The modified FOLFOX 6 regimen is as follows:

Oxaliplatin 85 mg/m², as a 2-hour infusion is administered every 2 weeks. Leucovorin (400 mg/m² in D5W) is administered every 2 weeks as a 2-hour infusion. Oxaliplatin is given at the same time as the leucovorin in a separate bag using a Y-line. The administration of leucovorin+oxaliplatin is followed by a 5-FU bolus of 400 mg/m² and then by 5-FU, 2400 mg/m² in D5W administered as a 46 hour continuous infusion.

Neuropathy assessment is performed at baseline and after every two cycles of therapy (approximately every month) by an independent neurologist. The subject and the neurologist are not informed whether the platinum infused is oxaliplatin or picoplatin. This assessment by the neurologist is used to determine the incidence of Grade 2 or greater peripheral neuropathy. In Phase 2, for the purpose of determining toxicity for dose reduction or study drug discontinuation, the treating physician performs a neurological assessment using the NCI CTCAE. These CTCAE criteria are used to determine the need to dose reduce prior to each cycle. The assessment of the neurologist is used for determination of the safety endpoint, the incidence of neuropathy, and is performed independently every other cycle using the protocol-specified neuropathy scale, but is not be used for dose modification. For all subjects, hematology and serum chemistry laboratory studies are obtained prior to each treatment cycle. Treatment cycles (5-FU and leucovorin±picoplatin or oxaliplatin depending on schedule) are repeated every 2 weeks, but may be delayed up to 2 weeks while awaiting recovery of clinical or laboratory abnormalities. Data from all cycles of treatment and cumulative toxicity are assessed for safety analysis.

Tumor evaluations will be done at baseline and after every 4th treatment of 5-FU/leucovorin (every 8 weeks, unless doses have been delayed) on study. The efficacy endpoint will include objective response rate according to RECIST criteria.²⁶ Duration of response, time to progression, progression-free survival, and overall survival are also evaluated.

The study treatments are summarized in Table 1, below:

TABLE 1

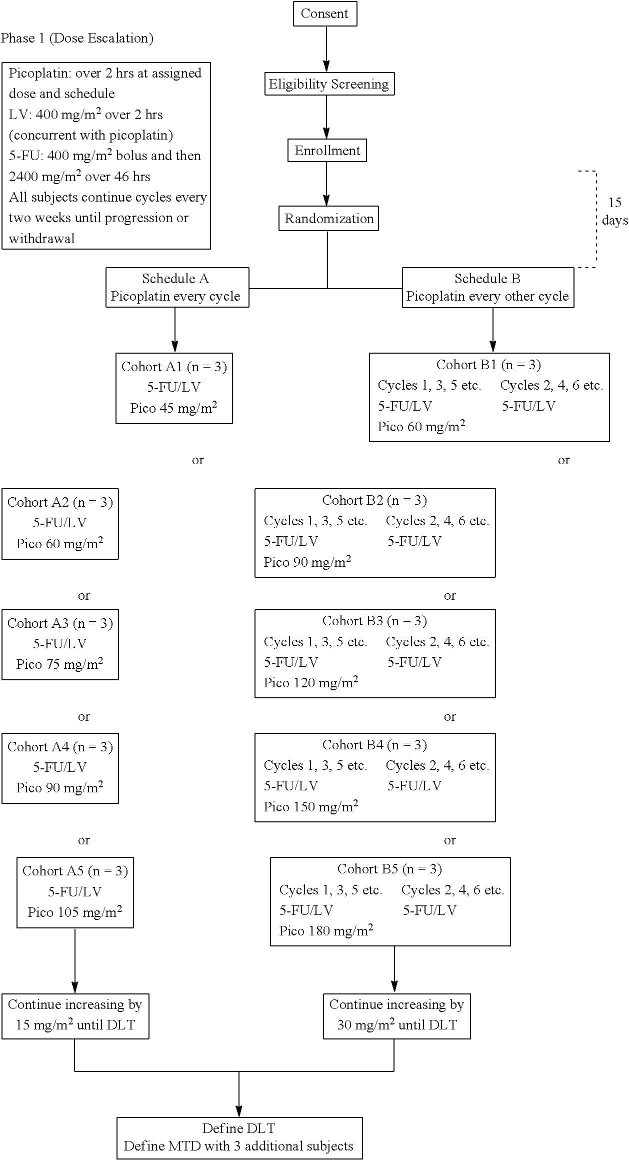

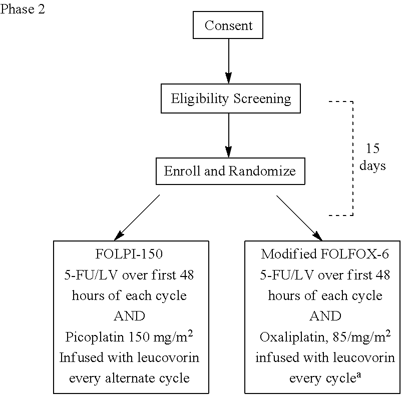

^{a}Picoplatin: over 2 hours 150 mg/m²; oxaliplatin: 85 mg/m², over 2 hours; LV:
400 mg/m² over 2 hours (concurrent with picoplatin when given or oxaliplatin)
followed by 5-FU: 400 mg/m² bolus and then 2400 mg/m² over 46 hours. All
subjects continue cycles every two weeks until progression or discontinuation of
study drug due to toxicity.

### Selection of Picoplatin Dose

Picoplatin was generally tolerated in combination with other myelosuppressive chemotherapeutic agents in previous Phase 1 studies at doses of 120-150 mg/m² administered every 3 weeks, i.e., doses equivalent to 80-100 mg/m² every 2 weeks or 160-200 mg/m² administered every 4 weeks. None of these studies, however, studied picoplatin in combination with 5-FU and leucovorin. 5-FU/leucovorin is not generally myelotoxic and thus the doses of picoplatin selected as the initial starting doses in the dose escalation portions of the current study, i.e., 45 mg/m² every two weeks and 60 mg/m² every four weeks, were well below the expected MTDs of picoplatin administered on these schedules.

### Administration of Picoplatin

Investigational-site staff must use standard cytotoxic handling procedures when preparing picoplatin for administration. Picoplatin is supplied as a ready-to-use formulation. The contents of the vials must be transferred to a suitable bag for administration. The compatibility of the formulation with typical infusion equipment has been assessed, and results have established compatibility with EVA infusion bags, PVC infusion tubing, and polypropylene syringes when the materials are protected from light. PVC infusion bags are not recommended for administration of picoplatin.

The compatibility of the formulation with typical administration sets has been assessed, and limits of acceptability have been set as 8 hours in a covered infusion bag. The product is highly sensitive to light and should not be exposed to ambient light for more than 1 hour without light protection. The bag must be protected from light during preparation and administration at the time of use.

There is no preservative or bacteriostatic agent present in the picoplatin formulation. Therefore, picoplatin must be transferred under aseptic conditions. The solution must be completely used or discarded within 8 hours of introduction into an infusion bag. As with all platinum complexes, contact with aluminum should be avoided.

Picoplatin should be administered by peripheral vein or central line; it must not be given by the intramuscular or subcutaneous route. The starting dose will be calculated based on the body surface area from the height and weight of the patient. If the patient's weight changes by more than 10%, the treating physician must recalculate the body surface area and amend the dose.

Picoplatin should be administered over 2 hours. It should be administered concurrently with leucovorin, in separate bags using a Y-line, when the two drugs are to be given on the same day. These two drugs have been tested and shown to be compatible when administered in this manner.

Subjects also received anti-emetic therapy consisting of a 5-HT₃ receptor antagonist plus dexamethasone 30 minutes prior to a dose of picoplatin. Subjects may also receive anti-emetic therapy for several days following treatment, which may include oral lorazepam, prochlorperazine, or equivalent for up to 7 days, as clinically indicated for breakthrough nausea and/or vomiting.

### Guidance for Administration

Detailed guidance for administration of 5-FU and leucovorin are provided in the product labels. Briefly, leucovorin 400 mg/m² IV infusion in D5W will be administered over 2 hours at the same time as picoplatin (if picoplatin is to be given on that day), in separate bags using a Y-line, followed by a bolus of 5-FU=400 mg/m² and then by 5-FU 2,400 mg/m² in D5W (recommended) administered as a 46-hour continuous IV infusion.

### Dose Modifications

### Dose Modification of Picoplatin

Dose-reduction is mandatory if any of the following hematological events are observed during the previous cycle: absolute neutrophil count (ANC) <0.5×10⁹/L for at least 5 days; absolute neutrophil count <1.0×10⁹/L complicated with Grade ≧2 fever (>38.5° C.); platelet count <25×10⁹/L; not reaching a platelet count≧100×10⁹/L and ANC≧15×10⁹/L by Day 15.

Dose reduction is also required for any treatment events involving any treatment-related Grade 3 toxicity, any Grade 4 toxicity, or any renal toxicity or neurotoxicities as described below.

For subjects receiving picoplatin every 2 weeks, the dose reduction should be 15 mg/m²; for subjects receiving picoplatin every 4 weeks the dose reduction should be 30 mg/m².

### Dose Reduction in the Event of Serum Creatinine Changes

Serum creatinine must be measured before every dose of picoplatin. For subjects with abnormal serum creatinine, the dose of picoplatin (but not 5-FU or leucovorin) must be modified according to the following table in Phase 1:

| | Dose modification | |
|---|---|---|
| | for q 2 week | Dose modification |
| | (Schedule A) | for q 4 week |
| Serum Creatinine | picoplatin | (Schedule B) |
| Value | subjects | picoplatin subjects |
| ≦institutional | recommended dose | recommended dose |
| ULN | | |
| >1.0 to 1.5 times | reduce by 25% | reduce by 25% |
| ULN | | |
| >1.5 to 2.0 times | reduce by 50% | reduce by 50% |
| ULN | | |
| >2.0 times ULN | discontinue | discontinue |
| | treatment with | treatment with |
| | picoplatin | picoplatin |

In Phase 2, the following dose reductions will be required for elevated serum creatinine:

| | Dose modification for |
|---|---|
| Serum creatinine | Phase 2 FOLPI subjects |
| ≦institutional ULN | recommended dose |
| >1.0 to 1.5 times ULN | reduce by picoplatin 30 mg/m² |
| >1.5 to 2.0 times ULN | reduce by picoplatin 60 mg/m² |
| >2.0 times ULN | discontinue treatment with |
| | picoplatin |

### Dose Modification in the Event of Neurotoxicity

The dose of picoplatin should be modified according to the CTCAE grade of toxicity and its duration as follows:

Duration of Toxicity

| | Resolves | |
|---|---|---|
| Toxicity | before next | Persistent |
| Grade | cycle | (present at start of next cycle) |
| Grade 1 | No change | Maintain picoplatin dose |
| Grade 2 | No change | Reduce picoplatin dose by 30 mg/m² |
| Grade 3 | Reduce | Discontinue picoplatin |
| | picoplatin dose | |
| | by 30 mg/m² | |

| | |
|---|---|
| Grade 4 | Discontinue picoplatin |

Up to three dose reductions of a 30 mg/m² may occur should toxicity not improve or worsen at a later cycle.

### Dose Modification of 5-FU

The first time the dose of picoplatin is reduced, the bolus dose of 5-FU should be omitted. The second time the dose of picoplatin is reduced, the infusional dose should be reduced by 600 mg/m². Once decreased, the reduced dose of 5-FU should be continued; i.e., the dose of 5-FU should not be subsequently increased.

If the platelet count or ANC count is Grade 1 or 2 at day 15 in a cycle with picoplatin, and the subject receives the alternate i.e., even numbered cycle that does not include picoplatin, the dose of 5-FU should not be reduced at this cycle. At the next treatment cycle, the doses of picoplatin and 5-FU should be reduced by one level. Dose modifications for Grade 3 or 4 non-hematological events must be made. Continue treatment only once toxicity has resolved to <Grade 3.

### Dose Modification of Leucovorin

There are no dose modifications for leucovorin, unless drug sensitivity is suspected because of a temporal relationship to the time of leucovorin administration.

### Results

59 patients have been treated to date in Phase 1. In the q 2 w schedule, 1 of 6 patients showed a DLT of Grade 4 thrombocytopenia and 3 of 6 patients, showed Grade 4 neutropenia at a picoplatin dose level of 105 mg/m². The q 2 w schedule is now being evaluated at 120 mg/m². In the q 4 w schedule, DLT was observed at 180 mg/m² in 2 of 6 patients. The MTD was therefore set at 150 mg/m² in the q 4 w schedule. Patients have received up to 24 cycles and the therapy was well tolerated.

For both schedules, dose delays were primarily from neutropenia or thrombocytopenia, with increased hematological toxicity observed at higher doses. Grade 3 non-hematological toxicities related to treatment include 1 coronary artery spasm following FU infusion, 1 picoplatin infusional allergic reaction, 1 stomatitis, 2 diarrhea, 1 azotemia. The cardiac and stomatitis events were attributed to the 5-FU component. No Grade 2 or higher neuropathy has been reported, even for four patients who have received a cumulative picoplatin dose of greater than about 900 mg/m², a surprising and unexpected result, particularly in view of a high incidence of moderate to severe neuropathy observed at comparable doses of oxaliplatin. This indicates that picoplatin can be safely administered with FU and LV without the dose limiting neuropathy associated with FOLFOX regimens.

In Schedule A (picoplatin q 2 week), the preferred dosage range is about 45-120 mg/m², e.g., doses of 45 to105 mg/m², e.g., 45 mg/m².

In Schedule B (picoplatin q 4 week), the preferred dose can be higher, e.g., about 120-210 mg/m², e.g., 120-180 mg/m², e.g., 150 mg/m². A lower dose can also be administered, e.g., at 45-90 mg/m², e.g., 60 mg/m².

Of 44 evaluated subjects evaluated by CT scan there have been 6 confirmed partial responses and one complete response (unconfirmed) (16%). Twenty-six of 32 subjects of the Q2 week schedule have been evaluated and 2 partial responses were observed. Surprisingly, ⅔ patients in cohort A1 (45 mg/m²) showed a partial response. Eighteen of 18 subjects in the Q4 week schedule have been evaluated and 5 partial responses were observed (28%).

### REFERENCES

The following references and other publications, patents and patent applications cited herein are incorporated by reference herein.
1. Jemal et al., Cancer Statistics, 2004. CA Cancer J Clin 54(1): 8-29, 2004.
2. Hoff et al., Oncology (Huntingt) 18(6): 705-708, 2004.
3. Meyerhardt et al., N Engl J Med 352(5): 476-87, 2005.
4. Penland et al., Oncology (Huntingt) 18(6): 715-722, 2004.
5. Saltz et al., N Engl J Med, 343(13): 905-14, 2000.
6. Tournigand et al., J Clin Oncol, 22(2): 229-37, 2004.
7. de Gramont et al., J Clin Oncol, 18(16): 2938-47, 2000.
8. Rothenberg et al., J Clin Oncol, 21(11): 2059-69, 2003.
9. Andre et al., N Engl J Med, 350(23): 2343-51, 2004.
10. Hwang et al., In: Clinical Use of Oxaliplatin: Case Studies and Roundtable Discussion, Editor Marshall J, CMP Healthcare Media, Oncology Publishing Group, Manhasset, N.Y. 2004.
11. Douillard, J Y, Schiller, J., Eur J Cancer 38(Suppl 8): S25-S31, 2002.
12. Beale, P, et al., Br J Cancer 88(7): 1128-1134, 2003.
13. Raynaud F I, et al., Clin Cancer Res 3(11): 2063-2074, 1997.
14. Holford J, et al., Anticancer Drug Des 13(1): 1-18, 1998.
15. Holford J, et al., Br J Cancer 77(3): 366-373, 1998.
16. Rogers P, et al., Eur J Cancer 38(12):1653-1660, 2002.
17. Sharp S Y, et al., Eur J Cancer 38(17):2309-15, 2002.
18. Plasencia C, et al., Invest New Drugs 22(4):399-409, 2004.
19. Murakami H, et al., Eur J Cancer 38(Suppl 8): S1-S5, 2002
20. Giaccone G, et al., Eur J Cancer 38 (Suppl 8): S19-S24, 2002.
21. Gore M E, et al., Eur J Cancer 38(18): 2416-2420, 2002.
22. Treat J, et al., Eur J Cancer 38(Suppl 8): S13-18, 2002.
23. Perez R P, et al., Eur J Cancer 34(10): 1535-42, 1998.
24. Gelmon K A, et al., Ann Oncol 15(7):1115-22, 2004.
25. Gelmon K A, et al., National Cancer Institute of Canada—Clinical Trials Group trial, IND 129. Ann Oncol 14: 543-548, 2003.
26. Therasse P, et al., New Guidelines to Evaluate the Response to Treatment in Solid Tumors. European Organization for Research and Treatment of Cancer, National Cancer Institute of the United States, National Cancer Institute of Canada. J Natl Cancer Inst 92(3): 205-216, 2000.
27. J. M. Tabernero et al., J. Clin. Oncol., 22(145), 3512 (2004).

Useful agents for administration with picoplatin and methods of treatment are also disclosed in include the platinum and non-platinum anticancer drugs disclosed in U.S. patent application Ser. No. 10/276,503, filed Sep. 4, 2003; Ser. No. 11/982,841, filed Nov. 5, 2007; Ser. No. 11/935,979, filed Nov. 6, 2007; Ser. No. 11/982,839, filed Nov. 5, 2007; in U.S. Pat. Nos. 7,060,808 and 4,673,668; in PCT WO/98/45331 and WO/96/40210.

The following patent applications are incorporated herein by reference in their entireties:

U.S. Ser. No. 61/027,387, filed Feb. 8, 2008, attorney docket no. 295.114prv

PCT Ser. No. ______, filed Feb. 6, 2009, attorney docket no. 295.114wol

U.S. Ser. No. 61/027,382, filed Feb. 8, 2008, attorney docket no. 295.115prv

U.S. Ser. No. 61/027,360, filed Feb. 8, 2008, attorney docket no. 295.116prv

PCT Ser. No. ______, filed Feb. 6, 2009, attorney docket no. 295.116wol

U.S. Ser. No. 11/982,841, filed Nov. 5, 2007, attorney docket no. 295.093usl

U.S. Ser. No. ______, filed Feb. 6, 2009, attorney docket no. 295.131usl

1. A method of treatment of colorectal cancer, comprising:
administering to a patient afflicted with colorectal cancer picoplatin, cetuximab, 5-fluorouracil (5-FU) and leucovorin, wherein 5-FU and leucovorin are administered intravenously at least twice at intervals of about 2-6 weeks, the picoplatin is administered with the leucovorin and 5-FU every other time that the fluorouracil and leucovorin are administered, and the cetuximab is administered at least twice at one-week intervals.

2. The method of claim 1, wherein the picoplatin is administered at a dose of about 60-180 mg/m².

3. The method of claim 1, wherein the interval of administration of the 5-FU and the leucovorin is about two weeks and the interval of administration of the picoplatin is about four weeks.

4. A method of treatment of colorectal cancer, comprising:
administering to a patient afflicted with colorectal cancer effective amounts of a combination of picoplatin, cetuximab, 5-FU and leucovorin, wherein the picoplatin, and the 5-FU and the leucovorin are administered intravenously at least twice at intervals of about 2-6 weeks, and the cetuximab is administered at least twice at one-week intervals, wherein an amount of picoplatin administered is less than the maximum tolerated dose of picoplatin.

5. The method of claim 4, wherein the picoplatin is administered at a dose of about 45-150 mg/m², preferably at a dose of about 135-150 mg/m².

6. The method of claim 4, wherein the interval of administration of the picoplatin, 5-FU and the leucovorin is about two weeks.

7. A method for selecting a treatment regimen for metastatic colorectal cancer (mCRC) comprising:
(a) providing a patient afflicted with mCRC;
(b) determining if the patient is a K-ras wild type mCRC patient; and
(c) if the patient comprises K-ras wild type mCRC, selecting for said patient a regimen comprising a EGFR inhibitor and picoplatin.

8. The method of claim 7 wherein the EGFR inhibitor is cetuximab or panitumumab.

9. The method of claim 7 or 8 wherein the regimen further comprises 5-FU and leucovorin.

10. The method of claim 7 or 8 wherein the patient is further treated with said regimen.

11. The method of any one of claims 1 or 4 wherein the cetuximab is administered intravenously at a first dose of about 250-500 mg/m², followed by doses of about 200-300 mg/m² administered at weekly intervals.

12. The method of any one of claims 1, 4 or 7 wherein the patient has not previously been treated for metastatic disease.

13. The method of claim 12 wherein the patient has previously been treated with an irinotecan, FOLFOX and/or FOLPI regimen.

14. The method of any one of claim 12 wherein the patient has previously been treated with a FOLFOX regimen, and subsequently with a FOLPI regimen and has relapsed within 6 months of completing the FOLPI regimen.

15. The method of any one of claims 1, 4 or 7 wherein the patient has previously been treated with a first regimen comprising FOLFOX or irinotecan, and subsequently with a second regimen comprising cetuximab alone, irinotecan plus cetuximab, or FOLPI, and has relapsed within 6 months following cessation of the second regimen.

16-21. (canceled)

22. The method of claim 4 wherein the picoplatin is administered substantially concurrently with the leucovorin followed by administration of the 5-FU at every treatment of the patient, and the cetuximab is administered at one week intervals.

23-24. (canceled)

25. The method of claim 1 or 4 wherein the picoplatin is administered at a dosage of about 120-150 mg/m².

26-30. (canceled)

31. The method of any one of claims 1 or 4 wherein the cetuximab is administered intravenously at a first dose of about 400 mg/m², then once a week at a dose of about 250 mg/m².

32. The method of claim 1 wherein the leucovorin, at a dosage of about 400 mg/m², is administered as a 2 hour infusion, the administration of the leucovorin being followed by a 5-FU bolus at a dosage of about 400 mg/m²; the 5-FU bolus being followed by 5-FU at a dosage of about 2,400 mg/m² administered as a 46 hour continuous infusion; wherein the leucovorin and the 5-FU are administered to the patient every 2 weeks and about 60-150 mg/m² of the picoplatin is administered to the patient with the leucovorin every 4 weeks, wherein at least the initial dose of picoplatin is about 150 mg/m², and wherein the cetuximab is administered at an initial dose of about 400 mg/m², then once a week at a dose of about 250 mg/m².

33. The method of any one of claims 1 or 4 further comprising, prior to administering the cetuximab, determining that the cancer of the patient is free of a K-ras mutation.

34-36. (canceled)

37. A method for selecting a regimen of treatment for a patient afflicted with a metastatic cancer that comprises EGFR, comprising (a) identifying a patient afflicted with a metastatic cancer, (b) determining if the cancer comprises a wild-type K-ras gene or a mutation-positive K-ras gene and (c) selecting a treatment regimen comprising picoplatin and an EGFR inhibitor if the wild type K-ras gene is present, or selecting a treatment regimen comprising picoplatin without an EGFR inhibitor if the mutation-positive K-ras gene is present.

38. The method of claim 37 wherein the metastatic cancer that comprises EGFR comprises SCLC, NSCLC, a pancreatic cancer, a colorectal cancer, an epithelial cancer, or a head and neck, ovarian, cervical, bladder, esophageal, gastric, breast, or endometrial cancer.

39. A method for selecting a regimen of treatment for a patient afflicted with mCRC comprising: (a) identifying a patient afflicted with mCRC, (b) determining if the mCRC comprises a wild type K-ras gene or a mutated K-ras gene and (c) if the m-CRC comprises a K-ras wild type genotype, then administering to the patient an EGFR inhibitors such as cetuximab, erlotinib or panitumumab, in combination with picoplatin and, optionally, 5-FU and leucovorin, or (d) if the mCRC comprises a K-ras mutation positive genotype, then administering to the patient picoplatin and, optionally, 5-FU and leucovorin.

40. The method of claim 39 wherein the EGFR inhibitor comprises cetuximab.

41-76. (canceled)
